(19) United States
(12) Patent Application Publication
(10) Pub. No.: US 2013/0035622 A1
(43) Pub. Date: Feb. 7, 2013

(54) TEMPORARY SPLINT

(71) Applicants: Patrick Schütz, Hamburg (DE); Koen Jozef Maria Jorissen, Hamburg (DE)

(72) Inventors: Patrick Schütz, Hamburg (DE); Koen Jozef Maria Jorissen, Hamburg (DE)

(73) Assignee: BSN medical GmbH, Hamburg (DE)

(21) Appl. No.: 13/434,048

(22) Filed: Mar. 29, 2012

(30) Foreign Application Priority Data
Aug. 4, 2011 (DE) 10 2011 109 431.1

(51) Int. Cl. A61F 5/00

(52) U.S. Cl. 602/6

(57) ABSTRACT

A splint to be placed on a section of a patient's body, that includes a body element having a bearing surface facing the body section, and an outer surface facing away from the bearing surface, formed from a flat, flexible material. The body element includes a first bearing section and a second bearing section arranged in relation to each other at a splint angle. The support body is elongate and has at least two elongate sections arranged at an angle corresponding to the splint angle between the bearing sections of the body element.

### TECHNICAL FIELD AND BACKGROUND OF THE INVENTION

The present invention relates to a splint for supporting a section of a patient's body by means of a body element consisting of a flat, flexible material that has bearing surface facing the body section and an outer surface facing away from the bearing surface.

Such a splint is known, for example, from EP 2 065 019 A1 wherein, according to a first exemplary embodiment described therein, the splint consists of a flat body that is bent such that the splint comprises a first body element and a second body element, each having a U-shaped cross-section, wherein the longitudinal axes of the body elements are perpendicular to each other, that is, they enclose a splint angle of 90′. Accordingly, an arm, for example, can be accommodated in the splint, wherein the elbow can extend out of the opening provided between the body elements.

In addition, another exemplary embodiment is disclosed in EP 2 065 019 A1 in which a body element comprises a first bearing section and a second bearing section that are arranged at an angle in relation to each other, wherein the material of the body element is, in turn, a flat, ductile, flexible material, and the angle between the bearing sections is established by an additional support body also formed from a flexible material.

The splints described in EP 2 065 019 A1 are associated with the advantage that the respective splint does not have to be transported in the state of its final shape but rather as a flat blank that is only unfolded directly before use. In the folded state, the splint takes up relatively little space which allows it to be transported or carried along in ambulances or the like.

However, such a splint has to be fastened to the corresponding body section of the patient using a bandage material. This results in the problem, which must be reliably prevented, of the bandage detaching from the body section while being worn, or the bandage material shifting in relation to the splint.

Another problem of a splint affixed with the assistance of bandage material is that the area between the splint and the section of the patient's body is not sufficiently ventilated, and wound exudate leaking from a wound cannot be easily drained. This can cause macerations to form on the patient.

In addition, there is the risk of the flexible material of the body element softening from sweat and wound exudate when it is made of cardboard or the like.

Another problem of the known splint is that inflammation can arise in the area of the supported body section that lies against the splint, especially when the material of the splint has already absorbed moisture.

Another problem with the known splint having two body elements arises from the fact that, when the splint is made of a blank formed from a single piece, an overlapping connection is created to connect the free ends of the blank. However, the risk at this location is that pressure points can arise on the supported body section in the area of the overlapping connection;

Another disadvantage of the known splint is that it is insufficiently stable to support the area of the shin and foot of a patient without becoming damaged when the patient steps on the floor. It was shown that the splint is insufficient in this regard. In addition, impact from walking is transferred undamped to the foot and shin, which is also undesirable.

When the body element of the splint has two hearing sections arranged at an angle in relation to each other, it is necessary to provide a support body that establishes the angle between the bearing sections. If this support body is also made of a flexible, flat material, it may not have the necessary rigidity, or the volume of the support body may have to be inappropriately large.

Finally, the flat material for the splint needs to be flexible in a preferred direction, whereas high flexural rigidity is desirable in a direction perpendicular thereto. In this context, it was shown that conventional cardboard material frequently does not satisfy these requirements. There is, therefore, a need to provide an improved material for the splints.

It is the overall object of the present invention to provide a splint of the initially cited type that does not have the aforementioned disadvantages.

### SUMMARY OF THE INVENTION

According to a first aspect of the present invention, the aforementioned object is achieved by a splint that is placed on a body section of a patient and comprises a body element which has a bearing surface facing the body section and an outer surface facing away from the bearing surface, and that is formed from a flat, flexible material, wherein the body element has a first bearing section and a second bearing section that are arranged at a split angle in comprises a relation to each other, and specifying support body for wherein the support body the splint angle, against the outer lies characterized in that support the support a rod surface, body is formed in the shape of corresponding to the splint angle between the bearing sections of the body element.

Alternatively, this solution can also be implemented by means of a splint that supports a body section of a patient and comprises a first body element and a second body element, wherein the body elements are formed from a flat, flexible, ductile material, wherein the first and second body element have a substantially U-shaped cross-section in a plane perpendicular to the longitudinal axis of the body element, wherein the longitudinal axes run in the middle of the U-shaped cross-section through the body elements, wherein the body elements have a bearing surface facing the inside of the U-shaped cross-section and an outer surface opposite the bearing surface, wherein the body elements are connected with each other such that the longitudinal axis of the first body element and the longitudinal axis of the second body element together enclose a splint angle, and comprises a support body for specifying the splint angle, wherein the support body lies against the outer surfaces of the body elements, characterized in that the support body is formed in the shape of a rod, and has at least two rod sections arranged at an angle such that they establish the splint angle.

In this context, the term “flat” within the meaning of the present invention is to be understood as a body whose largest dimension in a plane exceeds the dimension in a direction perpendicular to this plane by at least one order of magnitude and preferably by more than one order of magnitude.

The rod-shaped support body that establishes the angle between the longitudinal axes of the body elements or the bearing sections makes it unnecessary to use a folded support body of flexible material with a complex structure that may not ensure sufficient stability or may take up a large volume.

The support body is preferably formed from a thermoplastic with a softening temperature ranging from 50° C. to 200° C., preferably 60° C. to 170° C., and most preferably 70° C. to 140° C. The angle can then be specified by bending after simply heating the support body, and then the support body can be cooled so that the angle is fixed. A physician or paramedic can thereby easily adjust the desired angle without the manufacturer having to undertake special measures in this regard.

In a preferred embodiment, the support body is formed from a non-woven that is generated from cotton, polypropylene and polyester in an additionally preferred manner. It is particularly preferred when the non-woven comprises 40% cotton, 30% polypropylene and 30% polyester. These details refer to the percentage of weight of the non-woven. When the grammage of the non-woven from which the support body is formed is greater than 500 g/m², it has the necessary strength.

It is further advantageous when the support body has a connecting element that is connected to the first section and second section at a distance from the connection between them in order to establish the splint angle. The additional connecting element prevents the sections of the support element that lie on the bearing sections from being able to subsequently bend toward or away from each other during use and changing the splint angle.

It is also preferable when fastening devices are provided between the support body and outer surface of the body element(s) so that the support body is detachably connected thereto. This makes it easier to affix the splint.

A special embodiment of the fastening elements is designed such that fastening elements are provided on the side of the support body facing the outer surface that have a first section connected to the support element, extend from it, and have a first dimension perpendicular to its direction of extension, wherein at the end of the first section distant from the support body, a second section is provided that has a second dimension perpendicular to its direction of extension, wherein the second dimension is larger than the first dimension, and wherein openings assigned to the fasting elements are provided in the body element. In a particularly preferred manner, the fastening elements are formed from an elastic plastic, especially silicone.

Such fastening elements designed with a mushroom shape can be pressed into the openings in the body elements so that the support element can be thereby easily fastened.

Alternatively, it is also possible to fasten a support element by means of a hook/loop connection, which is a Velcro connection, to body elements.

According to another independently inventive aspect of the present invention, the aforementioned objective is achieved by a splint that supports a body section of a patient and comprises a body element consisting of a flat, flexible material having a bearing surface facing the body section, and an outer surface facing away from the bearing surface, wherein the bearing surface has an antimicrobial coating.

The antimicrobial coating significantly reduces the risk of inflammation on the patient's body section supported by the splint.

In a preferred embodiment, the antimicrobial coating can be designed as an acetate carrier, and this is laminated to the hearing surface in an additionally preferred manner. Such a coating is extremely efficient against pathogens.

As an alternative, it is also possible for the antimicrobial coating to have dialkyl carbamoyl chloride (DACC) and/or polyhexamethylene biguanide (PHMB) and/or silver as the active substance. The active substance can be incorporated in a nonwoven, woven fabric, knit or weave that is laminated to the bearing surface supported body section. Introducing the active substance into a non-woven, woven fabric, knit or weave makes it continuously available to the bearing surface so that the antimicrobial effect will exist over a long period of time. This effect exists, in particular, when the non-woven, woven fabric, knit or weave is provided with the active substance as early as the manufacturing stage.

According to another also independently inventive aspect of the present invention, the aforementioned objective is achieved by a splint that supports a body section of a patient and comprises a body element consisting of a flat, flexible material having a bearing surface facing the body section, and an outer surface facing away from the bearing surface, wherein an absorptive layer is applied to the bearing surface to absorb liquid and wound exudate.

The absorptive layer prevents wound exudate released by a wound in the area of the supported body section from penetrating the flexible material of the splint and possibly softening it; the exudate is instead stored by the absorptive layer. This prevents two problems. On the one hand, wound exudate is drained which reduces the danger of maceration in the patient; on the other hand, this ensures that the temporary splint cannot lose stability from released wound exudate.

The absorptive layer is preferably made of foam with cavities that serve to absorb the exudate. In particular, it can be open-cell polyurethane foam with a density that preferably lies between 30 and 110 kg/m³, and/or its thickness lies between 2.5 and 15 mm. For normal applications, such foam has a sufficiently high storage capacity and nevertheless does not become too bulky.

As an alternative to foam, the absorptive layer can be formed as a non-woven composite layer, wherein the non-woven composite layer has a layer of viscose and a polyester layer on the surface of the viscose layer facing away from the body element.

An absorptive layer having such a structure ensures that absorbed exudate will not be easily released which is guaranteed by the polyester layer on the side of the composite layer facing the body section.

According to another independently inventive aspect of the present invention, the aforementioned objective is achieved by a splint that supports a body section of a patient and comprises a body element consisting of a flat, flexible material having a bearing surface facing the body section, and an outer surface facing away from the bearing surface, wherein a layer is applied on at least part of the outer surface that has a fabric, especially bandage material, with greater static friction than the material of the body element.

The high static friction coefficient of the layer applied to the outer surface in relation to a bandage material ensures that it cannot slip once the splint has been fastened to the body section of the patient with the assistance of a bandage material. This ensures the attachment will be long-lasting.

Alternatively, the layer can be designed as a coating or laminated later.

It is particularly advantageous when the layer is designed as a film or a material proceeding in a grid pattern that is preferably formed from a polymer material. Such a design produces a favorable engagement between the layer and the bandage material so that the latter cannot shift in relation to the splint when it is under tension.

In an alternative embodiment, it is also possible to apply the hook element of a hook/loop connection, or a Velcro connection, to the outer surface that can also reliably affix the bandage material to the body element.

According to another independently inventive aspect of the present invention, the above object is achieved by means of a splint that supports a body section of a patient and comprises a first body element and a second body element, wherein the body elements are formed from a flat, flexible material, wherein the first and second body element have a substantially U-shaped cross-section in a plane perpendicular to the longitudinal axis of the body element, wherein the longitudinal axes run in the middle of the U-shaped cross-section through the body elements, and wherein the body elements are connected to each other such that the longitudinal axis of the first body element and the longitudinal axis of the second body element enclose a splint angle, characterized in that the connection between the first and second body element has two connecting elements that are fastened to outward facing surfaces of the body elements such that edges of the body elements oppose each other in a plane.

The connecting elements that connect the two body elements make it possible for the edges of the body elements facing each other to form a butt joint, the body elements hence proceeding in a common plane without an overlapping connection arising that could lead to pressure points in the area of the patient's body section supported by the splint.

Furthermore, the aforementioned object is achieved, in an independently inventive manner, by means of a splint that supports a body section of a patient and comprises a first body element and a second body element, wherein the body elements are formed from a flat, flexible material, wherein the first and second body elements have a substantially U-shaped cross-section in a plane perpendicular to the longitudinal axis of the body element, wherein the longitudinal axes run in the middle of the U-shaped cross-section through the body elements, and wherein the body elements are connected to each other such that the longitudinal axis of the first body element and the longitudinal axis of the second body element enclose a splint angle, characterized in that tabs are provided at the edges, which are distant from the longitudinal axis, of the first and/or second body element on both sides of the longitudinal axis, which extend out from the longitudinal axis.

The tabs extending from the outer edges of the body element can be connected to each other on the side of the body section facing away from the longitudinal axis such that the body section is completely enclosed by the body element, and the splint is reliably affixed to the body section. This is especially advantageous when the splint is to be used to support the upper and lower arm of a patient. The tabs can then be provided on the body element in which the upper arm is inserted such that the tabs then effectively fix the upper arm within the corresponding body element.

It is furthermore advantageous when the tabs proceed perpendicular to the longitudinal axis of the body element, since the reactive force arising when the free ends of the tabs are fastened to each other then proceeds in the direction of the extension of the tabs and no shearing force arises in them.

It is furthermore advantageous when the tabs have folding lines that extend parallel to the longitudinal axis of the body element since the tab can then be easily bent around the body section to be supported.

Given the fixation by the tabs, it is unnecessary for the body element to extensively surround the body section to be supported. Consequently, the outer edges of the body element can run at an angle to the longitudinal axis.

When the angle that encloses both longitudinal axes of the body elements, that is, the splint angle, is between 80° and 100°, and is preferably 90°, the splint can be employed quite effectively to support an arm or a foot joint.

Iris further advantageous when cutouts that preferably have an elongated shape are provided in the area of the edges of the body elements distant from the longitudinal axes, wherein the direction of the greatest extension runs parallel to the longitudinal axis of the body element. This makes it possible to affix the splint to the body section to be supported with the aid of belts, wherein the belts then extend through the cutouts.

Finally, it has proven to be advantageous when cuts arranged in the shape of a star or foam elements are provided in the area of the connection of the body elements. This prevents pressure points from arising in the area of the splint at which it lies against a joint.

According to another independently inventive aspect of the present invention, the aforementioned object is achieved by means of a splint that supports a body section of a patient and comprises a first body element and a second body element, wherein the body elements are formed from a flat, flexible material, wherein the first and second body element have a substantially U-shaped cross-section in a plane perpendicular to the longitudinal axis of the body element, wherein the longitudinal axes run in the middle of the U-shaped cross-section through the body elements, and wherein the body elements are connected to each other such that the longitudinal axis of the first body element and the longitudinal axis of the second body element enclose a splint angle, and wherein the body elements have a bearing surface facing the inside of the U-shaped cross-section and an outer surface opposite the bearing surface, characterized in that the first body element has a flat additional element extending parallel thereto.

By means of the, additional element that can be provided either on the bearing surface or the outer surface of the splint, the stability of the splint is enhanced in the area of this body element. This is particularly advantageous when one of the body elements lies on the sole of the foot, and the patient is supposed to walk with it despite the splint. Then increased stability in the area of this body element is necessary, which is ensured by this additional element.

When the additional element is provided on the outer surface, it is advantageous in such a case for it to be formed from an elastic material so that impact can be dampened while walking.

Finally, the initially cited object is achieved in an independently inventive manner by a splint comprising at least one first body element, wherein the body element is formed from a flat, flexible, ductile material, characterized in that the material has a first layer and a second layer that run parallel at a distance from each other, that strips are provided between the first and second layer, and that the strips meander along a path.

A splint designed in such a manner with a material having a meandering strip has high stability in the direction of the path of the strip, whereas it is nonetheless possible to bend the material perpendicular to the direction of the path.

Especially when the material of the strip is flat and extends perpendicular to the direction of the path of the first and second layers, the material is very rigid but has a low weight, which increases wearing comfort. The first and second layer can be formed from cardboard or polypropylene. It is, however, also conceivable for the first or second layer to be, a non-woven. To further increase stability, it is preferred to arrange straight strips between the meandering strips in the area between the layers.

In the following, the present invention will be explained with reference to drawings that only show preferred exemplary embodiments, wherein:

### BRIEF DESCRIPTION OF THE DRAWINGS

FIG. 1 shows a first perspective view of a first preferred exemplary embodiment of a splint according to the invention;

FIG. 2 shows a second perspective view of the first exemplary embodiment;

FIG. 3 shows a blank for the exemplary embodiment shown in FIGS. 1 and 2;

FIGS. 4a, 4b, and 4c show cross-sections of exemplary embodiments of different materials that can be used for the exemplary embodiment according to FIGS. 1 to 3, among other. things;

FIGS. 5 and 6 show supplements to the exemplary embodiment shown in FIGS. 1 to 3;

FIG. 7 shows a representation of an alternative exemplary embodiment to the exemplary embodiment shown in FIGS. 1 to 3;

FIG. 8 shows a perspective view of another alternative exemplary embodiment to the exemplary embodiment shown in FIGS. 1 to 3;

FIGS. 9a and 9b show representations of support elements for use in exemplary embodiments of splints according to FIGS. 1, 7 and 8;

FIG. 10 shows a detailed view of a support element from FIG. 9;

FIG. 11 shows a plan view of another exemplary embodiment of a splint according to the invention;

FIG. 12 shows a section along line XII-XII from FIG. 11;

FIGS. 13 and 14 show side views of support bodies for the exemplary embodiment according to FIG. 13 [sic];

FIG. 15 shows a cross-section of another embodiment of the material of the body elements of the exemplary embodiments; and

FIGS. 16 and 17 show a perspective view of exemplary embodiments of the material for the body elements of the exemplary embodiments.

### DETAILED DESCRIPTION OF THE PREFERRED EMBODIMENT AND BEST MODE

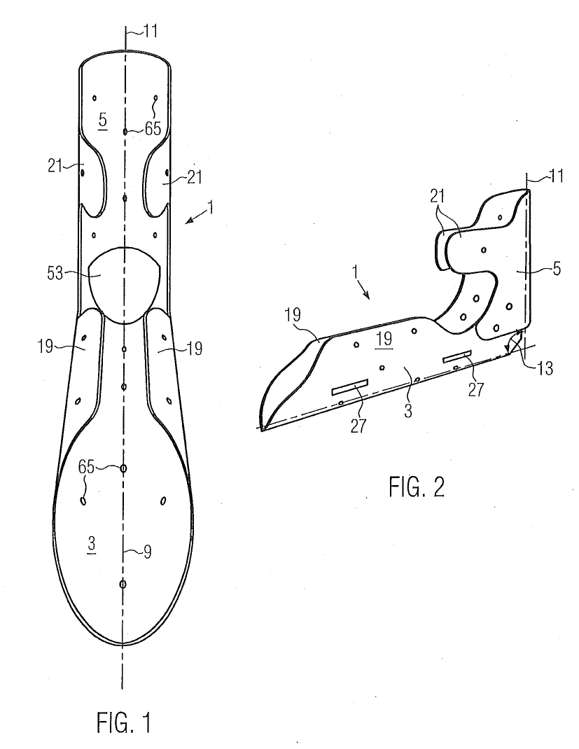

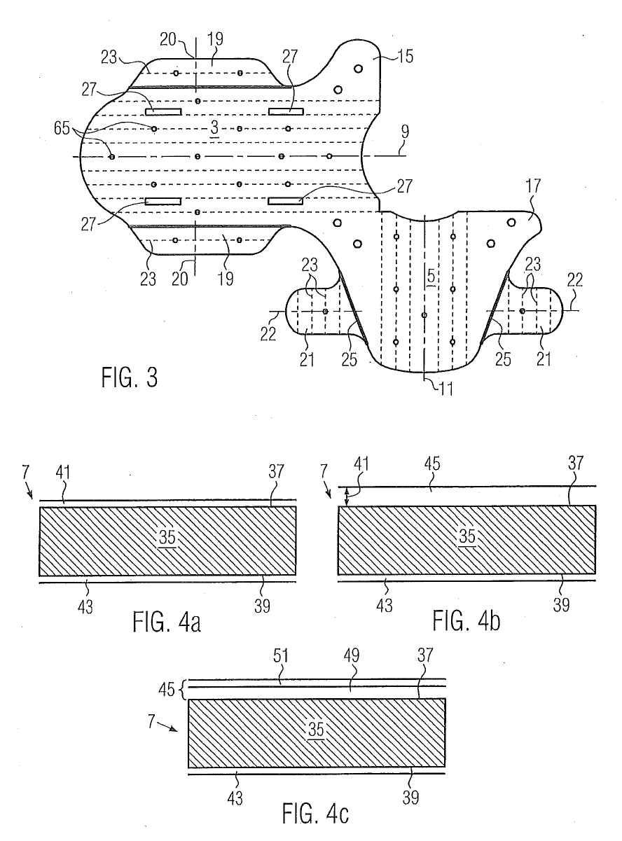

FIGS. 1 to 3 show a first exemplary embodiment of a splint 1 according to the invention, wherein FIG. 3 shows a blank from which the splint 1 shown in a perspective view in FIGS. 1 and 2 can be folded.

As can be seen in the figures, the first exemplary embodiment of a splint 1 according to the invention has a first body element 3 and a second body element 5 that are formed from a flat, flexible material 7. In the context of the present invention, the term “flat” material or body is to be understood as a material or body whose largest dimension in a plane in which the body proceeds exceeds the dimension in the direction perpendicular to this plane by at least one order of magnitude, and preferably by more than one order of magnitude. Consequently, cardboards or similar flat materials formed from plastic fall under this term.

As can also be seen in FIGS. 1 to 3, both the first and second body element 3, 5 are bent in a U-shape in a plane extending perpendicular to the longitudinal axis 9, 11 of the respective: body element 3, 5. The longitudinal axes 9, 11 defined in this manner run in the middle of the U-shaped cross-section through the body elements 3, 5. In addition, the body elements 3, 5 are connected to each other such that the longitudinal axes 9, 11 together enclose a splint angle 13 in a folded state, wherein the splint angle 13 is between 80° and 100°, and in the preferred exemplary embodiment shown here, is approximately 90°.

In the present exemplary embodiment shown in FIGS. 1 to 3, the body elements 3, 5 are connected such that when the blank shown in FIG. 3 is folded, the two free ends 15, 17 are connected to each other so that the U-shaped cross-section results for both body elements 3, 5 solely therefrom. It is, however, also conceivable for the body elements 3, 5 to be formed from separate elements instead of a single-piece blank such that the body elements 3, 5 then have to be connected to each other at two locations at a distance from each other. It is also possible in this context for the body elements 3, 5 to be pivoted with respect to each other such that the splint angle 13 can be adjusted.

As can also be seen in FIGS. 1 to 3, both the first body element 3 and second body element 5 have tabs 19, 21 on both sides of the longitudinal axis 9, 11 that extend therefrom on both sides of the longitudinal axis 9, 11. In the preferred exemplary embodiment shown here, both the tabs 19 on the first body element 3 as well as the tabs 21 on the second body element 5 extend along a respective direction of extension 20, 22 projecting perpendicular to the respective longitudinal axis 9, 11 and also have folding lines 23 extending parallel to the longitudinal axis 9, 11 so that the tabs can be easily bent parallel to the longitudinal axes 9, 11.

Furthermore, the second body element 5 is dimensioned such that its outer edges 25 extend at an angle to the longitudinal axis 11 of the second body element 5.

Furthermore, in the exemplary embodiment shown in FIGS. 1 to 3, cutouts 27 are provided in the first body element 3 at a distance from the longitudinal axis 9 and have an elongated shape and are arranged in a first body element 3 such that the direction of the greatest extension of the cutouts 27 runs parallel to the longitudinal axis 9 of the first body element 3. When the splint 1 is applied, belts (not shown) can be guided through these cutouts 27, which fasten the splint 1, for example, to an arm or leg section of a patient.

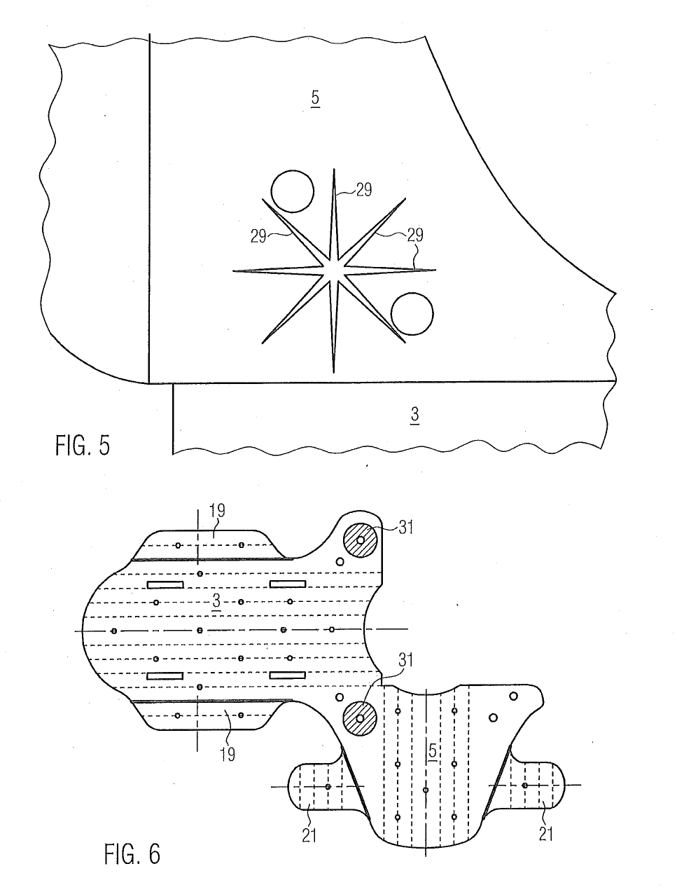

In addition, in deviation from the exemplary embodiment shown in FIGS. 1 to 3, cuts 29 can be provided in the area of the connection of the body elements 3, 5 that proceed in the shape of a star so that the material of the body elements 3, 5 can be easily bent outward, and the risk of pressure points can be avoided, especially on the joints of a supported patient's body section (see FIG. 5) the area of the connection of the body elements 3, 5 that then also come in contact with the body section in the area of a joint and also likewise avoid pressure points. This alternative is shown in FIG. 6.

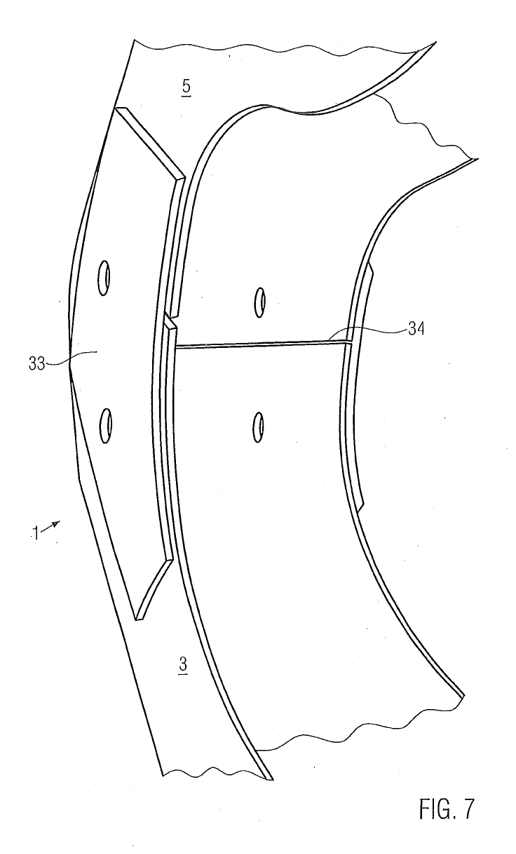

FIG. 7 shows the design of a splint 1 in which the body elements 3, 5 are separate from each other and not formed in a blank with a single part design as was the case with the exemplary embodiment shown in FIGS. 1 to 3. This exemplary embodiment also has body elements 3, 5 that are bent in a U-shape around a longitudinal axis 9, 11, and tabs 19, 21 are formed on their edges facing away from the longitudinal axis 9, 11 and extend perpendicular thereto. In addition, the body elements 3, 5 are connected to each other such that two connecting elements 33 are provided that are fastened to the outward-facing surface of the body elements 3, 5 so that edges of body elements 3, 5 facing one another lie opposite each other in a plane, that is, a butt joint 34 is formed. Consequently, the surfaces of the body elements 3, 5 facing inward also run in a common plane, and there are no stages that otherwise could lead to pressure points on the patient.

FIG. 4 shows cross-sections of exemplary embodiments of materials that can be used, in particular, for the exemplary embodiments of splints 1, shown in FIGS. 1 to 3 and 7. However, these materials can also be used for other splints made of flat material so that the use of these materials described in the following is not restricted to the splints otherwise described herein.

These materials 7 all have a flat, flexible basic body 35 that has a bearing surface 37 facing the body section when the splint is in a folded state, and an outer surface 39 facing away from the bearing surface 37. According to the exemplary embodiment of a material 7 shown in FIG. 4a), an antimicrobial coating 41 is applied to the bearing surface 37 that can be designed as an acetate carrier, which, for example, is laminated to the bearing surface 37.

Alternatively, the antimicrobial coating 41 can have dialkyl carbamoyl chloride (DACC) and/or polyhexamethylene biguanide (PHMB) and/or silver as the active substance. The active substance can be incorporated in a non-woven, fabric, knit or weave, which is then laminated to the bearing surface 37 as a coating 41. It is advantageous in this context when the nonwoven, fabric, knit or weave has already been equipped with the active substance.

On the outer surface 39 facing away from the bearing surface 37, a layer 43 is applied in all exemplary embodiments of the material to be used for a splint 1 shown in FIG. 4; said layer has greater static friction with a fabric, and especially bandage material, than the basic body 35. This layer 43 can be applied as a coating. It is, however, also conceivable for the layer 43 increasing the static friction to be laminated on.

In a particularly preferred exemplary embodiment, the layer 43 has a material running in a grid shape that is preferably formed from a polymer material which is associated with the advantage that a grid creates favorable engagement with a bandage material wrapped around the splint 1, which prevents the bandage material from slipping.

Finally, it is also possible for the layer 43 to be designed as a hook element of a hook/loop connection. Such a layer 43 that is only partially on the outer surface also yields a favorable grip between the splint 1 and bandage material.

FIGS. 4b and c show that an absorptive layer 45 is provided on the bearing surface 37 facing the body section of the base body 35, which serves to absorb wound exudate. According to the exemplary embodiment shown in FIG. 4b), the absorptive layer 45 is designed as a foam, wherein it is preferable to use an open-cell polyurethane foam with a density between 30 and 110 kg/m³, and/or with a thickness 47 between 2.5 and 15 mm. Such a selection yields an absorptive layer 45 with a capacity sufficient to absorb normally draining quantities of wound exudate.

In the exemplary embodiment shown in FIG. 4c), the absorptive layer is designed as a non-woven bandage that has a viscose layer 49 which is applied directly on the bearing surface 37, wherein this, in turn, is covered by a polyester layer 51. Such a construction ensures that exudate absorbed by the absorptive layer 45 and held in the viscose layer 49 is not released, which is effectively prevented by the polyester layer 51.

The previously described exemplary embodiments of splints 1, 1′ according to the invention can be used such that the arm or leg of the patient is accommodated by the splint 1, l′ in a manner such that, for example, the underarm lies in the first body element 3, while the upper arm is accommodated by the second body element 5. The elbow can then extend through the opening 53 provided between the body elements 3, 5, and the arm is firmly secured by the tabs 19, 21 that can be bent due to the folding lines. In addition, belts can be guided through the cutouts 27, which further secure the splints 1, 1′.

As an alternative to the belts, it is also possible to wind bandage material around the splint 1, wherein the layer 43 provided on the outer surface 39 of the body elements 3, 5 reliably prevents the bandage material from slipping.

If an anti-microbial coating 41 is provided on the bearing surface 37, this reliably prevents inflammation from developing on the patient's body section that is supported by the splint 1, 1′ when the splint 1, 1′ is worn for a long time.

Alternately, an absorptive layer 45 causes wound exudate released by wounds to be absorbed and held by this layer 45 so that the base body 35 of the material 7 of the splint 1, 1′ does not soften. In addition, this keeps macerations from being able to form on the patient.

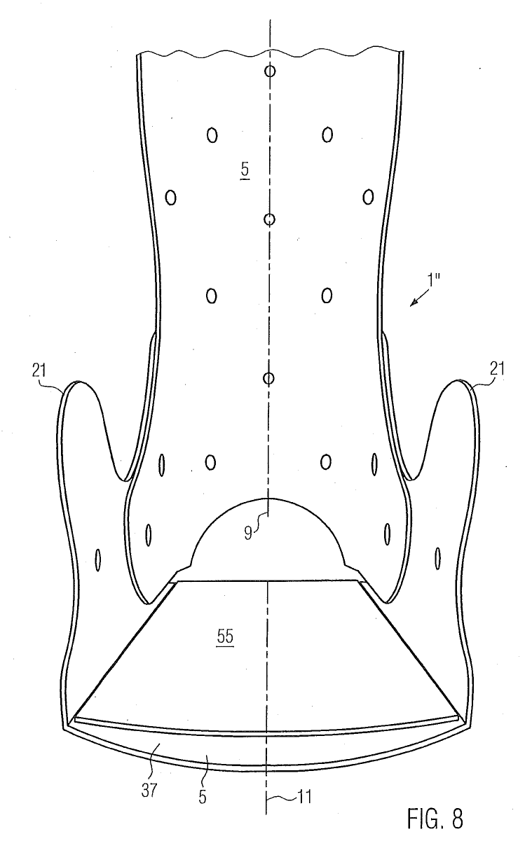

FIG. 8 shows an alternative to the exemplary embodiments shown in FIGS. 1 to 3 and 7, wherein the first body element 3 and the second body element 5 are designed as separate elements in this case that are not contained in a common blank designed as a single piece. In this case as well, the second body element 5 has tabs 21 that can be used in the present exemplary embodiment to secure the foot of a patient.

As can be seen in FIG. 8, an additional element 55 is provided on the bearing surface 37 of the second body element 5 that extends over a majority of the bearing surface 37 and serves to increase the stability of the second body element 5. This is beneficial, in particular, when the splint 1 is used to support a foot or shin, wherein the foot is accommodated in the second body element 5 and the user walks on the floor, which means that the second body element 5 has to absorb these loads. It is alternatively possible for the additional element 55 to be affixed on the outer surface and be made of an elastic material that can absorb impact.

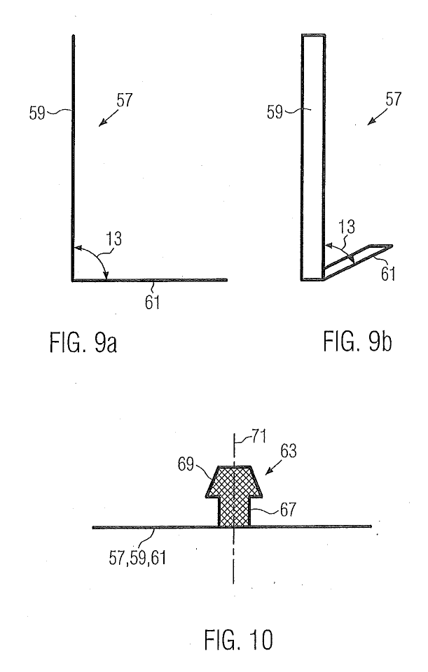

FIG. 9 shows a support body 57 in a side view (part a) and perspective view (part b) that can be used in a splint 1, 1′ as described with reference to FIGS. 1, 7 and 8. The support body 57 is designed in the shape of a rod and has a first rod section 59 and a second rod section 61, wherein the rod sections 59, 61 are arranged at an angle to each other.

The term “rod-shaped” according to the present invention includes bodies with dimensions that are at least one order of magnitude larger in a direction of extension defined by the body than the dimensions perpendicular to the direction of extension.

The support body 57 can abut the outer surface 39 of the body elements 3, 5 of the splint 1, 1′ and thereby establish the splint angle 13 assumed by the longitudinal axes 9, 11 of the body elements 3, 5. This is also important when the body elements 3, 5 are pivoted to each other such that the splint angle 13 can be freely adjustable.

The material of the support body 57 can be a thermoplastic material, and especially a thermoplastic with a softening temperature ranging between 50° C.′ and 200° C., preferably between 60° C. and 170° C., and most preferably between 70° C. and 140° C. The support body 57 can then be heated until it is deformable because the softening temperature has been exceeded such that the splint angle 13 can be adjusted. After it cools, the support body 57 is placed on the body elements 3, 5, which may be pivoted to each other, such that the hardened support body 57 establishes the splint angle 13.

This makes it easy for a paramedic or physician to establish the splint angle, and the splint is nevertheless highly stable due to the rigid support body 57.

Alternatively, the material of the support body 57 can also be a non-woven, wherein a mixture of 40% cotton, 30% polypropylene and 30% polyester is preferred (the percents refer to percent by weight). It is especially preferable to use a non-woven with a grammage greater than 500 g/m², and this produces a support body with excellent stability.

Finally, as can be seen in FIG. 10, fastening devices in the form of fastening elements 63 can be attached to the support body 57 on the side facing the outer surface 39 of the splint 1, 1′. The fastening elements 63 can engage with holes 65 in the body elements 3, 5 of the splint 1 and are designed such that they have a first section 67 that is connected to the support element 57 or a rod section 59, 61, and a second section 69 that is arranged on the end of the first section 67 distant from the support body 57 or rod section 59, 61. The first section 67 and the second section 69 have dimensions such that the first dimension of the first section 67 perpendicular to the direction of extension 71 of the fastening element is smaller than the second dimension of the second section 69 perpendicular to the direction of extension 71. Consequently, a fastening element 63 substantially in the shape of a mushroom is created, wherein the second section 69 can grip behind the body element 3, 5 when the fastening element 63 is extended through a hole 65 in a body element 3, 5 to secure the support body 57.

The support element 57 can thereby easily be fastened to the body element. It is preferable when the fasting element 63 is made of an elastic plastic such as silicone.

As an alternative to the above-described fastening elements 63, it is, however, also possible for both the support body 57 as well as the body elements 3, 5 to have elements of a hook/loop connection on the outer surface 39 so that the support element 57 can be fastened by means of a “Velcro connection” to the body elements 3, 5.

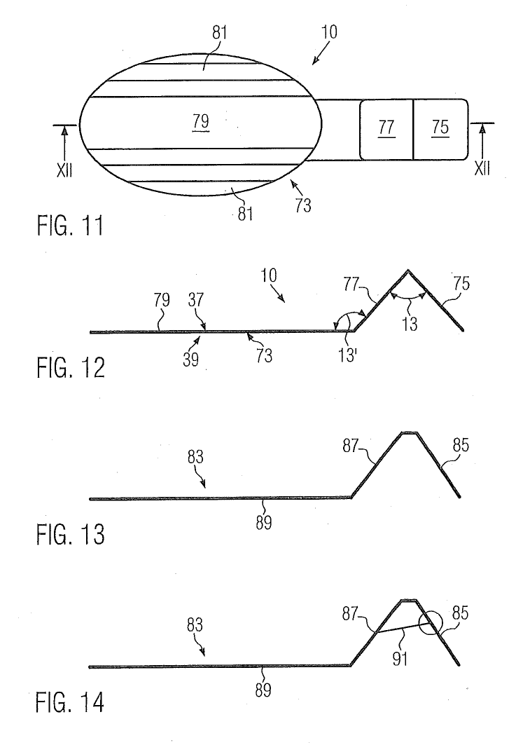

Another exemplary embodiment of a splint 10 according to the invention is shown in FIGS. 11 to 14, wherein the splint 10 has a flat body element 73 that has a first bearing section 75, a second bearing section 77 and a third bearing section 79 that can lie against a body section of a patient.

The material of the body element 73 can correspond to that described with reference to FIG. 4. It is also possible in this case to provide an antimicrobial coating 41, a layer 43 with elevated static friction or an absorptive layer 45.

The body element 73 is designed such that lateral tabs 81 are provided with folding lines 82 on the third bearing section 79, which run parallel to the direction extension of the body element 73 and can be bent upward due to the flexibility of the material of the body element 73. In addition, the splint 10 is designed such that a splint angle 13, 13′ is formed between the bearing sections 75, 77, 79, i.e., the sections do not run parallel to each other or in a single plane.

FIG. 13 shows a first example of a support body 83 of the splint 10 that is made of a rod-shaped material, which is rigid and only has a slight amount of elasticity. The support body comprises first, second and third rod sections 85, 87, 89 and is shaped such that the rod sections 85, 87, 89 are each arranged at an angle in relation to each other, wherein the angle between the first rod section 85 and second rod section 87 corresponds to the splint angle 13 between the first bearing section 75 and the second bearing section 77. In the same, manner, the angle between the second rod section 87 and third rod section 89 corresponds to the angle 13′ between the second bearing section 77 and third bearing section 79.

When in a mounted state, the support body 83 lies against the outer surface 39 of the body element 73 facing away from the bearing surface 37, which means that the support body 83 dictates the splint angle 13 and the angle 13′ between the second and third bearing section 77, 79.

In this case as well, the support body 83 can also be made of a thermoplastic with a softening temperature between 50° C. and 200° C., preferably between 60° C. and 170° C., and especially preferably between 70° C. and 140° C., so that the angles 13, 13′ can be easily adjusted by heating the support body 83. Consequently, the physician or paramedic has a great deal of flexibility when adjusting the splint with the splint 10 portrayed here. In this case as well, the support body 83 can be made of a mixture of non-woven consisting of cotton, polypropylene and polyester, wherein preferably 40% cotton, 30% polypropylene and 30% polyester are mixed (indicated as percent by weight) connected to the first and second rod section 85, 87 at a distance, in each instance, from the point where the rod sections 85, 87 are connected to each other. In this manner, the angle between the rod sections 85, 87 and hence the splint angle are reliably established by the connecting element 91.

Finally, in this exemplary embodiment as well, the support body 83 and the body element 73 can either be connected to the support body 83 via the fastening elements 63 shown in FIG. 10 and corresponding holes in the body element 73. Alternatively, a hook/loop connection can also be provided between the two. This enables a releasable connection between the body element 73 and support body 83 such that the body element 73 can be transported and stored in a space saving manner in the form of a flat blank.

The splint 10 can, in particular, be applied to the underarm of a patient in a manner such that the first and second bearing section 75, 77 support the palm, while the underarm itself lies on the third bearing section 89, wherein the support body 83 dictates the angle between the bearing sections 75, 77, 83. When an antimicrobial coating 41 or an absorptive layer 45 are provided, inflammation or a softening of the material of the body element as well as macerations on the patient are prevented when the splint 10 is secured by means of a bandage material.

An additional embodiment of a material for the base body (see FIG. 4) for the body elements 3, 5, 73 is shown in FIG. 15, wherein the material is flat and flexible and has a first and second layer 93, 95. The layers 93, 95 extend in the plane of the body element 3, 5, 73 and form the bearing surface 37 or the outer surface 39. The layers 93, 95 can be made of cardboard or polypropylene. However, other materials such as films are also conceivable. It is also possible for the first and second layer 93, 95 to be formed from a non-woven.

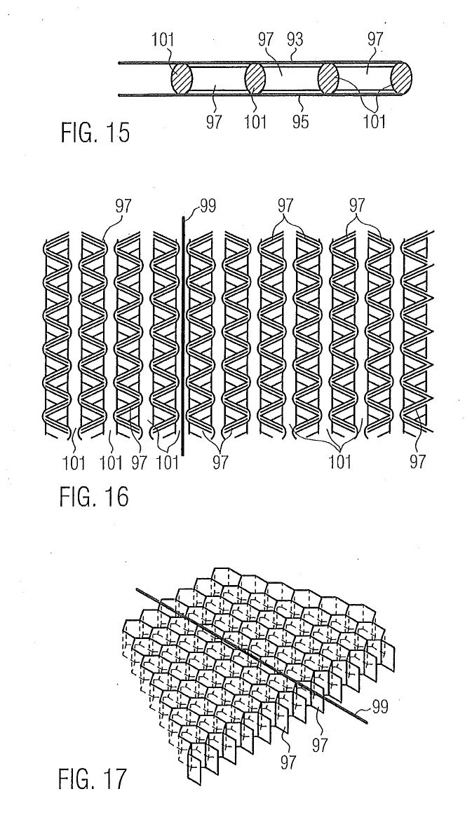

Strips are arranged between the first and second layer 93, 95 and are shown in a perspective view in FIG. 16. First strips 97 meander between the layers 93, 95 along a path 99. A honeycomb-like structure is formed by the first strips. In addition to the meandering first strips 97, straight, second strips 101 are also provided along the path 99 that further stabilize the material.

Such a structure of the material makes the body elements 3, 5, 73 very flexible along lines that run parallel to the path 99, while the body elements, however, have very high flexural rigidity in the direction of the path 99.

FIG. 17 shows an additional exemplary embodiment of the material of the body elements 3, 5, 73, wherein, in contrast to what is shown in FIG. 16, no straight strips 101 are provided, only meandering strips 97 that extend perpendicular to the first and second layer 93, 95, which means that a honeycomb-like structure also results in this case in the gap between the layers 93, 95. This material also has high flexural rigidity in the direction of the path 99 of the first strips 97, while the material can be easily bent in the direction perpendicular thereto.

As can be seen from the above, the aforementioned disadvantages of the prior art are eliminated by the splints 1, 1′ 10 according to the invention.

The foregoing description provides embodiments of the invention by way of example only. It is envisioned that other embodiments may perform similar functions and/or achieve similar results. Any and all such equivalent embodiments and examples are within the spirit and scope of the present invention and are intended to be covered by the appended claims.

I claim:

1. A splint to be placed on a section of a patient's body comprising:
(a) a body element (73) formed from a flat, flexible material and having a bearing surface (37) facing the body section, and an outer surface. (39) facing away from the bearing surface, (37);
(b) the body element (73) including a first bearing section (75) and a second bearing section (77), which are arranged in relation to each other at a splint angle (13); and
(c) the support body (83) being elongate and having at least two elongate sections (85, 87) arranged at an angle (13) corresponding to the splint angle (13) between the bearing sections (75, 77) of the body element (73).

2. The splint according to claim 1, wherein the material of the support body (57, 83) is a thermoplastic material with a softening temperature preferably ranging between 50° C. and 200° C., more preferably between 60° C. and 170° C., and most preferably between 70° C. and 140° C.

3. The splint according to claim 1, wherein the material of the support body (57, 83) is a non-woven material.

4. The splint according to claim 3, wherein the non-woven material comprises a mixture of cotton, polypropylene and polyester.

5. The splint according to claim 4, wherein the non-woven material comprises a mixture of 40% cotton, 30% polypropylene and 30% polyester.

6. The splint according to claim 5, wherein the weight of the non-woven material is greater than 500 g/m2.

7. The splint according to claim 1, wherein the support body (83) comprises a connecting element (91) that is connected to the first support body section (85) and second support body section (87) at a predetermined distance from the connection, where the first support section (85) and the second support section (87) are connected to each other and form the splint angle (13) between the support body sections (85, 87).

8. The splint according to claim 1, wherein complementary fastening devices (63, 65) are provided between the side of the support body (57, 83) to releasably connect the support element (57, 83) to the body element (3, 5, 73).

9. The splint according to claim 8, wherein fastening elements (63) are provided having a first section (67), connected to the support element (57, 83) and extending therefrom, and having a first dimension perpendicular to its direction of extension (71), wherein, on the end of the first section (67) distant from the support body (57, 83), a second section (69) is provided that has a second dimension perpendicular to its direction of extension (71), further wherein the second dimension is larger than the first dimension, and still further wherein openings (65) associated with the fastening devices (63) are provided in the body element (3, 5, 73).

10. The splint according to claim 9, wherein the fastening devices (63) are made of an elastic plastic.

11. The splint according to claim 8, wherein the fastening elements comprise a hook element and a complementary loop element,

12. The splint according to claim 1, wherein the body element (73) is formed from a flat, flexible material having a first layer (93) and a second layer (95) spaced-apart and parallel in relation to each other, and are connected to each other by interconnecting strips (97).

13. The splint according to claim 12, wherein the interconnecting strips (97) are flat, and extend perpendicular to the first and second layer (93, 95).

14. The splint according to claim 12 or 13, wherein at least one of the first and second layer (93, 95) is made from a material selected from the group consisting of cardboard and polypropylene.

15. The splint according to claim 12 or 13, wherein the first and/or second layer (93, 95) comprise a non-woven material.

16. The splint according to claim 12, wherein second strips (101) are provided that are arranged in a straight line path (99) between the strips (97) that extend perpendicular to the first and second layer (93, 95).

17. The splint according to claim 1, wherein the splint is a lower leg splint.

18. The splint according to claim 1, wherein the splint is a foot and ankle splint.

19. The splint according to claim 1, wherein the splint is a lower arm splint.

20. The splint according to claim 1, wherein the splint is a hand/wrist splint.
